(19) United States
(12) Patent Application Publication
(10) Pub. No.: US 2022/0380674 A1
(43) Pub. Date: Dec. 1, 2022

(54) Photoplethysmographic Sensor Containing A Polymer Composition

(71) Applicant: Ticona LLC, Florence, KY (US)

(72) Inventors: Young Shin Kim, Cincinnati, OH (US); Philip T. Wilson, Cincinnati, OH (US); Don DeMello, Salado, TX (US)

(21) Appl. No.: 17/732,585

(22) Filed: Apr. 29, 2022

Related U.S. Application Data
(60) Provisional application No. 63/189,950, filed on May 18, 2021.

(51) Int. Cl. C09K 19/38; C09K 19/06; C09K 19/34; A61B 5/024; A61B 5/00

(52) U.S. Cl. CPC C09K 19/3804; C09K 19/061; C09K 19/067; C09K 19/3444; A61B 5/02416; A61B 5/02438; A61B 5/681; A61B 5/6831; A61B 5/6843; A61B 5/746; C09K 2019/521

(57) ABSTRACT

A photoplethysmographic sensor comprising a light source for emitting light onto a tissue and an optical detector for receiving light that interacts with the tissue is provided. The sensor comprises a liquid crystalline polymer.

### RELATED APPLICATION

The present application is based upon and claims priority to U.S. Provisional Patent Application Ser. No. 63/189,950, having a filing date of May 18, 2021, which is incorporated herein by reference.

### BACKGROUND OF THE INVENTION

Most soft tissue will transmit and reflect both visible and near-infrared radiation. Thus, if light is projected onto an area of skin and the reflected light detected after its interaction with the skin, blood, and other tissues, time varying changes in light absorbance can be observed. PPG sensors are often employed for this purpose that include one or more light sources (e.g., LEDs) that emit light onto the skin and one or more optical detectors (e.g., photodiodes) that receive the reflected light. Notably, the time varying light absorbance signal (photoplethysmographic or “PPG” signal) may be affected by a number of factors, some of which include the optical properties of the tissues and blood at the measurement site, and the wavelength of the light source. PPG sensors may thus be used in applications to determine various physiological factors, such as heart rate and oxygen saturation determination. The quality of the PPG signal in such a sensor, however, may be affected by a variety of other factors, including non-pulsatile signal artifacts and noise. The manner in which the PPG sensor is employed may also reduce the quality of the PPG signal. For example, when the PPG sensor is employed in a wearable device, the user's motion and movement can also reduce the quality of the PPG signal. Of course, various steps can be taken to try and reduce signal noise, but ultimately it would be desirable if the PPG sensor itself could be configured in such a way that the signal noise had a minimal impact on the detection of the PPG signal.

### SUMMARY OF THE INVENTION

In accordance with one embodiment of the present invention, a photoplethysmographic sensor is disclosed that comprises a light source for emitting light onto a tissue and an optical detector for receiving light that interacts with the tissue. The sensor comprises a liquid crystalline polymer.

Other features and aspects of the present invention are set forth in greater detail below.

### BRIEF DESCRIPTION OF THE FIGURES

A full and enabling disclosure of the present invention, including the best mode thereof to one skilled in the art, is set forth more particularly in the remainder of the specification, including reference to the accompanying figures, in which:

FIG. 1A is a schematic illustration of one embodiment of a wearable device that may employ the PPG sensor of the present invention;

FIG. 1B is a block diagram showing the components of the wearable device of FIG. 1A;

FIG. 2 is a schematic illustration of one embodiment of the PPG sensor of the present invention; and

FIG. 3 is a schematic illustration of another embodiment of the PPG sensor of the present invention.

### DETAILED DESCRIPTION

It is to be understood by one of ordinary skill in the art that the present discussion is a description of exemplary embodiments only, and is not intended as limiting the broader aspects of the present invention.

Generally speaking, the present invention is directed to a photoplethysmographic (“PPG”) sensor. The PPG sensor generally includes at least one light source that is capable of emitting light into or onto a user's tissue (e.g., skin, blood, etc.) and at least one optical detector that receives light that reflects the light after it interacts with the tissue. To obtain physiological information (e.g., heart rate, oxygen saturation, etc.), a PPG signal can be generated from the received light, which is a time varying change in light absorbance. To help minimize the distortion of the measured light and signal loss due to movement, one or more components of the sensor are generally formed from a polymer composition that includes a liquid crystalline polymer.

The polymer composition may be formed to have a melt viscosity that is sufficiently low to enable it to be readily molded into the small dimensions required for a PPG sensor. For example, the polymer composition may have a melt viscosity of about 200 Pa-s or less, in some embodiments about 150 Pa-s or less, in some embodiments about 100 Pa-s or less, in some embodiments from about 5 Pa-s to about 90 Pa-s, and in some embodiments, from about 10 to about 70 Pa-s, as determined in accordance with ISO Test No. 11443:2014 at a shear rate of 400 seconds⁻¹ at a temperature of about 30° C. above the melting temperature (e.g., about 380° C.). Conventionally, it was believed that polymer compositions exhibiting such a low melt viscosity would not also possess sufficiently good thermal and mechanical properties to enable good physical integrity for use in forming a PPG sensor. Contrary to conventional thought, however, the present inventors have discovered through careful control of the particular liquid crystalline polymer(s) and/or other optional materials, the resulting polymer composition can also possess both excellent thermal and mechanical properties. More particularly, the polymer composition typically has a melting temperature of about 280° C. or more, in some embodiments about 300° C. or more, in some embodiments about 320° C. or more, and in some embodiments, from about 330° C. to about 450° C., such as determined in accordance with ISO 11357-2:2013. Even at such melting temperatures, the ratio of the deflection temperature under load (“DTUL”), a measure of short-term heat resistance, to the melting temperature may still remain relatively high, which can, among other things, allow the use of high-speed processes for forming the PPG sensor. For example, the ratio may range from about 0.5 to about 1.00, in some embodiments from about 0.65 to about 0.95, and in some embodiments from about 0.75 to about 0.85. The specific DTUL values may, for instance, be about 160° C. or more, in some embodiments from about 200° C. to about 350° C., in some embodiments from about 220° C. to about 320° C., and in some embodiments from about 250° C. to about 300° C., such as determined in accordance with ISO Test No. 75-2:2013 (technically equivalent to ASTM D648-07) at a load of 1.8 Megapascals.

The polymer composition may be generally stiff in nature so that it is capable of maintaining the desired degree of physical integrity during formation of the PPG sensor. Such stiffness may be generally characterized by a high tensile modulus. For example, the tensile modulus may be about 8,000 MPa or more, in some embodiments about 10,000 MPa or more, in some embodiments about about 11,000 MPa or more, in some embodiments from about 12,000 MPa to about 30,000 MPa, in some embodiments from about 13,000 MPa to about 25,000 MPa, and in some embodiments, from about 14,000 MPa to about 22,000 MPa, such as determined in accordance with ISO Test No. 527:2019 at 23° C. The composition may also exhibit a tensile strength of from about 150 MPa or more, in some embodiments from about 160 to about 400 MPa, and in some embodiments, from about 170 to about 350 MPa and/or a tensile break strain of about 1% or more, in some embodiments about 2% or more, in some embodiments about 3% or more, in some embodiments from about 4% to about 20%, and in some embodiments, from about 5% to about 15%, such as determined in accordance with ISO Test No. 527:2019 at 23° C. The polymer composition may also exhibit a flexural modulus of about 10,000 MPa or more, in some embodiments about 11,000 MPa or more, in some embodiments from about 12,000 MPa to about 30,000 MPa, and in some embodiments, from about 13,000 MPa to about 25,000 MPa; a flexural strength of from about 40 to about 500 MPa, in some embodiments from about 100 to about 400 MPa, and in some embodiments, from about 150 to about 350 MPa; and/or a flexural break strain of about 0.5% or more, in some embodiments from about 1% to about 15%, and in some embodiments, from about 2% to about 10%, such as determined in accordance with ISO Test No. 178:2019 at 23° C. The composition may also exhibit a Charpy unnotched impact strength of about 45 kJ/m² or more, in some embodiments from about 45 to about 100 kJ/m², and in some embodiments, from about 50 to about 80 kJ/m², measured at 23° C. according to ISO Test No. 179-1:2010. The polymer composition may also exhibit excellent surface properties. The polymer composition may, for instance, exhibit a Rockwell surface hardness of about 65 or less, in some embodiments about 60 or less, and in some embodiments, from about 40 to about 55, as determined in accordance with ASTM D785-08 (2015) (Scale M).

In addition to possessing good mechanical, thermal, flow, and/or surface properties, the polymer composition may also exhibit good electrical properties to help reduce the amount of electrical noise encountered by the PPG sensor, particularly at the relatively high frequencies often used for wireless communication (e.g., Bluetooth, LTE, or 5G). For example, the polymer composition may exhibit a low dissipation factor (measure of the loss rate of energy), such as about 0.01 or less, in some embodiments about 0.009 or less, in some embodiments about 0.008 or less, in some embodiments, about 0.007 or less, in some embodiments about 0.006 or less, and in some embodiments, from about 0.001 to about 0.005 at high frequencies (e.g., 2 or 10 GHz). The dielectric constant may likewise be selectively tuned based on the presence of other optional materials in the composition. In certain embodiments, for example, the polymer composition may exhibit a low dielectric constant, such as about 6 or less, in some embodiments about 5 or less, in some embodiments from about 0.1 to about 4 and in some embodiments, from about 0.5 to about 3.5, and in some embodiments, from about 1 to about 3 at high frequencies (e.g., 2 or 10 GHz). In other embodiments, the polymer composition may exhibit a high dielectric constant, such as greater than about 6, in some embodiments about 8 or more, in some embodiments from about 8 to about 30, and in some embodiments, from about 10 to about 20, and in some embodiments, from about 1 to about 3 at high frequencies (e.g., 2 or 10 GHz). The polymer composition may also provide a high degree of shielding effectiveness to electromagnetic interference (“EMI”). More particularly, the EMI shielding effectiveness may be about 20 decibels (dB) or more, in some embodiments about 25 dB or more, and in some embodiments, from about 30 dB to about 100 dB, as determined in accordance with ASTM D4935-18 at high frequencies (e.g., 2 or 10 GHz).

Various embodiments of the present invention will now be described in more detail.

### I. Polymer Composition

A. Liquid Crystalline Polymer

Liquid crystalline polymers are generally classified as “thermotropic” to the extent that they can possess a rod-like structure and exhibit a crystalline behavior in their molten state (e.g., thermotropic nematic state). Such polymers typically have a melting temperature of about 280° C. or more, in some embodiments about 300° C. or more, in some embodiments about 320° C. or more, and in some embodiments, from about 330° C. to about 450° C. The polymers may be formed from one or more types of repeating units as is known in the art. The liquid crystalline polymer may, for example, contain one or more aromatic ester repeating units generally represented by the following Formula (I):

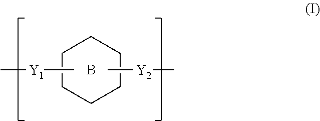

wherein,

ring B is a substituted or unsubstituted 6-membered aryl group (e.g., 1,4-phenylene or 1,3-phenylene), a substituted or unsubstituted 6-membered aryl group fused to a substituted or unsubstituted 5- or 6-membered aryl group (e.g., 2,6-naphthalene), or a substituted or unsubstituted 6-membered aryl group linked to a substituted or unsubstituted 5- or 6-membered aryl group (e.g., 4,4-biphenylene); and

Y₁ and Y₂ are independently O, C(O), NH, C(O)HN, or NHC(O).

Typically, at least one of Y₁ and Y₂ are C(O). Examples of such aromatic ester repeating units may include, for instance, aromatic dicarboxylic repeating units (Y₁ and Y₂ in Formula I are C(O)), aromatic hydroxycarboxylic repeating units (Y₁ is O and Y₂ is C(O) in Formula I), as well as various combinations thereof.

Aromatic hydroxycarboxylic repeating units, for instance, may be employed that are derived from aromatic hydroxycarboxylic acids, such as, 4-hydroxybenzoic acid; 4-hydroxy-4′-biphenylcarboxylic acid; 2-hydroxy-6-naphthoic acid; 2-hydroxy-5-naphthoic acid; 3-hydroxy-2-naphthoic acid; 2-hydroxy-3-naphthoic acid; 4′-hydroxyphenyl-4-benzoic acid; 3′-hydroxyphenyl-4-benzoic acid; 4′-hydroxyphenyl-3-benzoic acid, etc., as well as alkyl, alkoxy, aryl and halogen substituents thereof, and combination thereof. Particularly suitable aromatic hydroxycarboxylic acids are 4-hydroxybenzoic acid (“NBA”) and 6-hydroxy-2-naphthoic acid (“HNA”). When employed, repeating units derived from hydroxycarboxylic acids (e.g., HBA and/or HNA) typically constitute about 20 mol. % or more, in some embodiments about 25 mol. % or more, in some embodiments about 30 mol. % or more, in some embodiments about 40 mol. % or more, in some embodiments about 50 mol. % or more, in some embodiments from about 55 mol. % to 100 mol. %, and in some embodiments, from about 60 mol. % to about 95 mol. % of the polymer.

Aromatic dicarboxylic repeating units may also be employed that are derived from aromatic dicarboxylic acids, such as terephthalic acid, isophthalic acid, 2,6-naphthalenedicarboxylic acid, diphenyl ether-4,4′-dicarboxylic acid, 1,6-naphthalenedicarboxylic acid, 2,7-naphthalenedicarboxylic acid, 4,4′-dicarboxybiphenyl, bis(4-carboxyphenyl)ether, bis(4-carboxyphenyl)butane, bis(4-carboxyphenyl)ethane, bis(3-carboxyphenyl)ether, bis(3-carboxyphenyl)ethane, etc., as well as alkyl, alkoxy, aryl and halogen substituents thereof, and combinations thereof. Particularly suitable aromatic dicarboxylic acids may include, for instance, terephthalic acid (“TA”), isophthalic acid (“IA”), and 2,6-naphthalenedicarboxylic acid (“NDA”). When employed, repeating units derived from aromatic dicarboxylic acids (e.g., IA, TA, and/or NDA) each typically constitute from about 1 mol. % to about 40 mol. %, in some embodiments from about 2 mol. % to about 30 mol. %, and in some embodiments, from about 5 mol. % to about 25 mol. % of the polymer.

Other repeating units may also be employed in the polymer. In certain embodiments, for instance, repeating units may be employed that are derived from aromatic diols, such as hydroquinone, resorcinol, 2,6-dihydroxynaphthalene, 2,7-dihydroxynaphthalene, 1,6-dihydroxynaphthalene, 4,4′-dihydroxybiphenyl (or 4,4′-biphenol), 3,3′-dihydroxybiphenyl, 3,4′-dihydroxybiphenyl, 4,4′-dihydroxybiphenyl ether, bis(4-hydroxyphenyl)ethane, etc., as well as alkyl, alkoxy, aryl and halogen substituents thereof, and combinations thereof. Particularly suitable aromatic diols may include, for instance, hydroquinone (“HQ”) and 4,4′-biphenol (“BP”). When employed, repeating units derived from aromatic diols (e.g., HQ and/or BP) typically constitute from about about 1 mol. % to about 50 mol. %, in some embodiments from about 1 mol. % to about 40 mol. %, in some embodiments from about 2 mol. % to about 40 mol. %, in some embodiments from about 5 mol. % to about 35 mol. %, and in some embodiments, from about 5 mol. % to about 25 mol. % of the polymer.

Repeating units may also be employed, such as those derived from aromatic amides (e.g., acetaminophen (“APAP”)) and/or aromatic amines (e.g., 4-aminophenol (“AP”), 3-aminophenol, 1,4-phenylenediamine, 1,3-phenylenediamine, etc.). When employed, repeating units derived from aromatic amides (e.g., APAP) and/or aromatic amines (e.g., AP) typically constitute from about 0.1 mol. % to about 20 mol. %, in some embodiments from about 0.5 mol. % to about 15 mol. %, and in some embodiments, from about 1 mol. % to about 10 mol. % of the polymer. It should also be understood that various other monomeric repeating units may be incorporated into the polymer. For instance, in certain embodiments, the polymer may contain one or more repeating units derived from non-aromatic monomers, such as aliphatic or cycloaliphatic hydroxycarboxylic acids, dicarboxylic acids, diols, amides, amines, etc. Of course, in other embodiments, the polymer may be “wholly aromatic” in that it lacks repeating units derived from non-aromatic (e.g., aliphatic or cycloaliphatic) monomers.

In certain embodiments, the liquid crystalline polymer may be a “high naphthenic” polymer to the extent that it contains a relatively high content of repeating units derived from naphthenic hydroxycarboxylic acids and naphthenic dicarboxylic acids, such as NDA, HNA, or combinations thereof. That is, the total amount of repeating units derived from naphthenic hydroxycarboxylic and/or dicarboxylic acids (e.g., NDA, HNA, or a combination of HNA and NDA) is typically about 10 mol. % or more, in some embodiments about 12 mol. % or more, in some embodiments about 15 mol. % or more, in some embodiments about 18 mol. % or more, in some embodiments about 30 mol. % or more, in some embodiments about 40 mol. % or more, in some embodiments about 45 mol. % or more, in some embodiments 50 mol. % or more, in some embodiments about 55 mol. % or more, and in some embodiments, from about 55 mol. % to about 95 mol. % of the polymer. Without intending to be limited by theory, it is believed that such “high naphthenic” polymers are capable of reducing the tendency of the polymer composition to absorb water, which can aid in processability and the enhancement of physical properties. Namely, such high naphthenic polymers typically have a water adsorption of about 0.015% or less, in some embodiments about 0.01% or less, and in some embodiments, from about 0.0001% to about 0.008% after being immersed in water for 24 hours in accordance with ISO 62-1:2008. The high naphthenic polymers may also have a moisture adsorption of about 0.01% or less, in some embodiments about 0.008% or less, and in some embodiments, from about 0.0001% to about 0.006% after being exposed to a humid atmosphere (50% relative humidity) at a temperature of 23° C. in accordance with ISO 62-4:2008.

In one embodiment, for instance, the repeating units derived from HNA may constitute 30 mol. % or more, in some embodiments about 40 mol. % or more, in some embodiments about 45 mol. % or more, in some embodiments 50 mol. % or more, in some embodiments about 55 mol. % or more, and in some embodiments, from about 55 mol. % to about 95 mol. % of the polymer. In such embodiments, the liquid crystalline polymer may contain various other monomers, such as aromatic hydroxycarboxylic acid(s) (e.g., HBA) in an amount of from about 1 mol. % to about 50 mol. %, and in some embodiments from about 1 mol. % to about 20 mol. %, and in some embodiments, from about 2 mol. % to about 10 mol. %; aromatic dicarboxylic acid(s) (e.g., IA and/or TA) in an amount of from about 1 mol. % to about 40 mol. %, and in some embodiments, from about 5 mol. % to about 25 mol. %; and/or aromatic diol(s) (e.g., BP and/or HQ) in an amount of from about 1 mol. % to about 40 mol. %, and in some embodiments, from about 5 mol. % to about 25 mol. %. In another embodiment, the repeating units derived from NDA may constitute 10 mol. % or more, in some embodiments about 12 mol. % or more, in some embodiments about 15 mol. % or more, and in some embodiments, from about 18 mol. % to about 95 mol. % of the polymer. In such embodiments, the liquid crystalline polymer may also contain various other monomers, such as aromatic hydroxycarboxylic acid(s) (e.g., HBA) in an amount of from about 20 mol. % to about 60 mol. %, and in some embodiments, from about 30 mol. % to about 50 mol. %; aromatic dicarboxylic acid(s) (e.g., IA and/or TA) in an amount of from about 2 mol. % to about 30 mol. %, and in some embodiments, from about 5 mol. % to about 25 mol. %; and/or aromatic diol(s) (e.g., BP and/or HQ) in an amount of from about 2 mol. % to about 40 mol. %, and in some embodiments, from about 5 mol. % to about 35 mol. %.

Of course, “low naphthenic” liquid crystalline polymers may also be employed in the composition, either alone or in combination with “high naphthenic” liquid crystalline polymers. In such low naphthenic polymers, the total amount of repeating units derived from naphthenic hydroxycarboxylic and/or dicarboxylic acids (e.g., NDA, HNA, or a combination of HNA and NDA) is typically less than 10 mol. %, in some embodiments about 8 mol. % or less, in some embodiments about 6 mol. % or less, and in some embodiments, from about 1 mol. % to about 5 mol. % of the polymer.

Regardless of the particular constituents and nature of the polymer, the liquid crystalline polymer may be prepared by initially introducing the aromatic monomer(s) used to form the ester repeating units (e.g., aromatic hydroxycarboxylic acid, aromatic dicarboxylic acid, etc.) and/or other repeating units (e.g., aromatic diol, aromatic amide, aromatic amine, etc.) into a reactor vessel to initiate a polycondensation reaction. The particular conditions and steps employed in such reactions are well known, and may be described in more detail in U.S. Pat. No. 4,161,470 to Calundann; U.S. Pat. No. 5,616,680 to Linstid, III, et al.; U.S. Pat. No. 6,114,492 to Linstid, III, et al.; U.S. Pat. No. 6,514,611 to Shepherd, et al.; and WO 2004/058851 to Waggoner. The vessel employed for the reaction is not especially limited, although it is typically desired to employ one that is commonly used in reactions of high viscosity fluids. Examples of such a reaction vessel may include a stirring tank-type apparatus that has an agitator with a variably-shaped stirring blade, such as an anchor type, multistage type, spiral-ribbon type, screw shaft type, etc., or a modified shape thereof. Further examples of such a reaction vessel may include a mixing apparatus commonly used in resin kneading, such as a kneader, a roll mill, a Banbury mixer, etc.

If desired, the reaction may proceed through the acetylation of the monomers as known the art. This may be accomplished by adding an acetylating agent (e.g., acetic anhydride) to the monomers. Acetylation is generally initiated at temperatures of about 90° C. During the initial stage of the acetylation, reflux may be employed to maintain vapor phase temperature below the point at which acetic acid byproduct and anhydride begin to distill. Temperatures during acetylation typically range from between 90° C. to 150° C., and in some embodiments, from about 110° C. to about 150° C. If reflux is used, the vapor phase temperature typically exceeds the boiling point of acetic acid, but remains low enough to retain residual acetic anhydride. For example, acetic anhydride vaporizes at temperatures of about 140° C. Thus, providing the reactor with a vapor phase reflux at a temperature of from about 110° C. to about 130° C. is particularly desirable. To ensure substantially complete reaction, an excess amount of acetic anhydride may be employed. The amount of excess anhydride will vary depending upon the particular acetylation conditions employed, including the presence or absence of reflux. The use of an excess of from about 1 to about 10 mole percent of acetic anhydride, based on the total moles of reactant hydroxyl groups present is not uncommon.

Acetylation may occur in in a separate reactor vessel, or it may occur in situ within the polymerization reactor vessel. When separate reactor vessels are employed, one or more of the monomers may be introduced to the acetylation reactor and subsequently transferred to the polymerization reactor. Likewise, one or more of the monomers may also be directly introduced to the reactor vessel without undergoing pre-acetylation.

In addition to the monomers and optional acetylating agents, other components may also be included within the reaction mixture to help facilitate polymerization. For instance, a catalyst may be optionally employed, such as metal salt catalysts (e.g., magnesium acetate, tin(I) acetate, tetrabutyl titanate, lead acetate, sodium acetate, potassium acetate, etc.) and organic compound catalysts (e.g., N-methylimidazole). Such catalysts are typically used in amounts of from about 50 to about 500 parts per million based on the total weight of the recurring unit precursors. When separate reactors are employed, it is typically desired to apply the catalyst to the acetylation reactor rather than the polymerization reactor, although this is by no means a requirement.

The reaction mixture is generally heated to an elevated temperature within the polymerization reactor vessel to initiate melt polycondensation of the reactants. Polycondensation may occur, for instance, within a temperature range of from about 250° C. to about 380° C., and in some embodiments, from about 280° C. to about 380° C. For instance, one suitable technique for forming the aromatic polyester may include charging precursor monomers and acetic anhydride into the reactor, heating the mixture to a temperature of from about 90° C. to about 150° C. to acetylize a hydroxyl group of the monomers (e.g., forming acetoxy), and then increasing the temperature to from about 280° C. to about 380° C. to carry out melt polycondensation. As the final polymerization temperatures are approached, volatile byproducts of the reaction (e.g., acetic acid) may also be removed so that the desired molecular weight may be readily achieved. The reaction mixture is generally subjected to agitation during polymerization to ensure good heat and mass transfer, and in turn, good material homogeneity. The rotational velocity of the agitator may vary during the course of the reaction, but typically ranges from about 10 to about 100 revolutions per minute (“rpm”), and in some embodiments, from about 20 to about 80 rpm. To build molecular weight in the melt, the polymerization reaction may also be conducted under vacuum, the application of which facilitates the removal of volatiles formed during the final stages of polycondensation. The vacuum may be created by the application of a suctional pressure, such as within the range of from about 5 to about 30 pounds per square inch (“psi”), and in some embodiments, from about 10 to about 20 psi.

Following melt polymerization, the molten polymer may be discharged from the reactor, typically through an extrusion orifice fitted with a die of desired configuration, cooled, and collected. Commonly, the melt is discharged through a perforated die to form strands that are taken up in a water bath, pelletized and dried. In some embodiments, the melt polymerized polymer may also be subjected to a subsequent solid-state polymerization method to further increase its molecular weight. Solid-state polymerization may be conducted in the presence of a gas (e.g., air, inert gas, etc.). Suitable inert gases may include, for instance, include nitrogen, helium, argon, neon, krypton, xenon, etc., as well as combinations thereof. The solid-state polymerization reactor vessel can be of virtually any design that will allow the polymer to be maintained at the desired solid-state polymerization temperature for the desired residence time. Examples of such vessels can be those that have a fixed bed, static bed, moving bed, fluidized bed, etc. The temperature at which solid-state polymerization is performed may vary, but is typically within a range of from about 250° C. to about 350° C. The polymerization time will of course vary based on the temperature and target molecular weight. In most cases, however, the solid-state polymerization time will be from about 2 to about 12 hours, and in some embodiments, from about 4 to about 10 hours.

B. Other Additives

In some cases, liquid crystalline polymers may constitute the entire polymer composition (e.g., 100 wt. %). Nevertheless, it may be desirable in certain embodiments to include one or more additives within the polymer composition to help achieve certain target properties. In such embodiments, the polymer composition typically contains one or more liquid crystalline polymers in an amount of from about 30 wt. % to about 99 wt. %, in some embodiments from about 40 wt. % to about 95 wt. %, and in some embodiments, from about 50 wt. % to about 90 wt. % of the entire polymer composition, as well as one or more additives in an amount of from about 1 wt. % to about 70 wt. %, in some embodiments from about 5 wt. % to about 60 wt. %, and in some embodiments, from about 10 wt. % to about 50 wt. % of the polymer composition. When employed, the particular nature of the additives may vary, such as described in more detail below.

i. Mineral Filler

In certain embodiments, the polymer composition may contain a mineral filler, which may be in the form of particles (e.g., platelet-shaped, flake-shaped, etc.), fibers (or “whiskers”), and so forth. Typically, the mineral filler has a certain hardness value to help improve the mechanical strength, adhesive strength, and surface properties of the composition, which enables the composition to be uniquely suited to form the small parts of a PPG sensor. For instance, the hardness values may be about 2.0 or more, in some embodiments about 2.5 or more, in some embodiments about 3.0 or more, in some embodiments from about 3.0 to about 11.0, in some embodiments from about 3.5 to about 11.0, and in some embodiments, from about 4.5 to about 6.5 based on the Mohs hardness scale. Any of a variety of different types of mineral particles may generally be employed, such as those formed from a natural and/or synthetic silicate mineral, such as talc, mica, halloysite, kaolinite, illite, montmorillonite, vermiculite, palygorskite, pyrophyllite, calcium silicate, aluminum silicate, wollastonite, etc.; sulfates; carbonates; phosphates; fluorides, borates; and so forth. Particularly suitable are particles having the desired hardness value, such as calcium carbonate (CaCO₃, Mohs hardness of 3.0), copper carbonate hydroxide (Cu₂CO₃(OH)₂, Mohs hardness of 4.0); calcium fluoride (CaFl₂, Mohs hardness of 4.0); calcium pyrophosphate ((Ca₂P₂O₇, Mohs hardness of 5.0), anhydrous dicalcium phosphate (CaHPO₄, Mohs hardness of 3.5), hydrated aluminum phosphate (AlPO₄.2H₂O, Mohs hardness of 4.5); potassium aluminum silicate (KAlSi₃O₈, Mohs hardness of 6), copper silicate (CuSiO₃.H₂O, Mohs hardness of 5.0); calcium borosilicate hydroxide (Ca₂B₅SiO₉(OH)₅, Mohs hardness of 3.5); calcium sulfate (CaSO₄, Mohs hardness of 3.5), barium sulfate (BaSO₄, Mohs hardness of from 3 to 3.5), mica (Mohs hardness of 2.5-5.3), and so forth, as well as combinations thereof. Mica, for instance, is particularly suitable. Any form of mica may generally be employed, including, for instance, muscovite (KAl₂(AlSi₃)O₁₀(OH)₂), biotite (K(Mg,Fe)₃(AlSi₃)O₁₀(OH)₂), phlogopite (KMg₃(AlSi₃)O₁₀(OH)₂), lepidolite (K(Li,Al)₂₋₃(AlSi₃)O₁₀(OH)₂), glauconite (K,Na)(Al,Mg,Fe)₂(Si,Al)₄O₁₀(OH)₂), etc. Muscovite-based mica is particularly suitable for use in the polymer composition.

In certain embodiments, the mineral particles, such as barium sulfate and/or calcium sulfate particles, may have a shape that is generally granular or nodular in nature. In such embodiments, the particles may have a median size (e.g., diameter) of from about 0.5 to about 20 micrometers, in some embodiments from about 1 to about 15 micrometers, in some embodiments from about 1.5 to about 10 micrometers, and in some embodiments, from about 2 to about 8 micrometers, such as determined using laser diffraction techniques in accordance with ISO 13320:2020 (e.g., with a Horiba LA-960 particle size distribution analyzer). In other embodiments, it may also be desirable to employ flake-shaped mineral particles, such as mica particles, that have a relatively high aspect ratio (e.g., average diameter divided by average thickness), such as about 4 or more, in some embodiments about 8 or more, and in some embodiments, from about 10 to about 500. In such embodiments, the average diameter of the particles may, for example, range from about 5 micrometers to about 200 micrometers, in some embodiments from about 8 micrometers to about 150 micrometers, and in some embodiments, from about 10 micrometers to about 100 micrometers. The average thickness may likewise be about 2 micrometers or less, in some embodiments from about 5 nanometers to about 1 micrometer, and in some embodiments, from about 20 nanometers to about 500 nanometers such as determined using laser diffraction techniques in accordance with ISO 13320:2020 (e.g., with a Horiba LA-960 particle size distribution analyzer).

Suitable mineral fibers may likewise include those that are derived from silicates, such as neosilicates, sorosilicates, inosilicates (e.g., calcium inosilicates, such as wollastonite; calcium magnesium inosilicates, such as tremolite; calcium magnesium iron inosilicates, such as actinolite; magnesium iron inosilicates, such as anthophyllite; etc.), phyllosilicates (e.g., aluminum phyllosilicates, such as palygorskite), tectosilicates, etc.; sulfates, such as calcium sulfates (e.g., dehydrated or anhydrous gypsum); mineral wools (e.g., rock or slag wool); and so forth. Particularly suitable are fibers having the desired hardness value, including fibers derived from inosilicates, such as wollastonite (Mohs hardness of 4.5 to 5.0), which are commercially available from Nyco Minerals under the trade designation Nyglos® (e.g., Nyglos® 4W or Nyglos® 8). The mineral fibers may have a median width (e.g., diameter) of from about 1 to about 35 micrometers, in some embodiments from about 2 to about 20 micrometers, in some embodiments from about 3 to about 15 micrometers, and in some embodiments, from about 7 to about 12 micrometers. In addition to possessing the size characteristics noted above, the mineral fibers may also have a relatively high aspect ratio (average length divided by median width) to help further improve the mechanical properties and surface quality of the resulting polymer composition. For example, the mineral fibers may have an aspect ratio of from about 2 to about 100, in some embodiments from about 2 to about 50, in some embodiments from about 3 to about 20, and in some embodiments, from about 4 to about 15. The volume average length of such mineral fibers may, for example, range from about 1 to about 200 micrometers, in some embodiments from about 2 to about 150 micrometers, in some embodiments from about 5 to about 100 micrometers, and in some embodiments, from about 10 to about 50 micrometers.

When employed, mineral fillers typically constitute from about 5 to about 150 parts, in some embodiments from about 20 to about 100 parts, and in some embodiments, from about 40 to about 80 parts by weight per 100 parts by weight of liquid crystalline polymers employed in the composition. For example, mineral fillers may constitute from about 5 wt. % to about 60 wt. %, in some embodiments from about 15 wt. % to about 55 wt. %, and in some embodiments, from about 25 wt. % to about 50 wt. % of the polymer composition.

ii. Electrically Conductive Filler

If desired, an electrically conductive filler may be employed so that the polymer composition is generally antistatic in nature. More particularly, the polymer composition may exhibit a controlled resistivity that allows it to remain generally antistatic in nature such that a substantial amount of electrical current does not flow through the part, but nevertheless exhibits a sufficient degree of electrostatic dissipation to facilitate the ability of the composition to be plated if so desired. The surface resistivity may, for instance, range from about 1×10¹² ohms to about 1×10¹⁸ ohms, in some embodiments from about 1×10¹³ ohms to about 1×10¹⁸ ohms, in some embodiments from about 1×10¹⁴ ohms to about 1×10¹⁷ ohms, and in some embodiments, from about 1×10¹⁵ ohms to about 1×10¹⁷ ohms, such as determined in accordance with ASTM D257-14 (technically equivalent to IEC 62631-3-1). Likewise, the composition may also exhibit a volume resistivity of from about 1×10¹⁰ ohm-m to about 1×10¹⁶ ohm-m, in some embodiments from about 1×10¹¹ ohm-m to about 1×10¹⁶ ohm-m, in some embodiments from about 1×10¹² ohm-m to about 1×10¹⁵ ohm-m, and in some embodiments, from about 1×10¹³ ohm-m to about 1×10¹⁵ ohm-m, such as determined at a temperature of about 20° C. in accordance with ASTM D257-14 (technically equivalent to IEC 62631-3-1).

To achieve the desired degree of antistatic behavior, a single material may be selected having the desired resistivity, or multiple materials may be blended together (e.g., insulative and electrically conductive) so that the resulting filler has the desired resistivity. In one particular embodiment, for example, an electrically conductive material may be employed that has a volume resistivity of less than about 1 ohm-cm, in some embodiments about less than about 0.1 ohm-cm, and in some embodiments, from about 1×10⁻⁸ ohm-cm to about 1×10⁻² ohm-cm, such as determined at a temperature of about 20° C. in accordance with ASTM D257-14 (technically equivalent to IEC 62631-3-1). Suitable electrically conductive carbon materials may include, for instance, graphite, carbon black, carbon fibers, graphene, carbon nanotubes, etc. Other suitable electrically conductive fillers may likewise include metals (e.g., metal particles, metal flakes, metal fibers, etc.), ionic liquids, and so forth. In one embodiment, for instance, the antistatic filler may be an ionic liquid. One benefit of such a material is that, in addition to being an antistatic agent, the ionic liquid can also exist in liquid form during melt processing, which allows it to be more uniformly blended within the polymer matrix. This improves electrical connectivity and thereby enhances the ability of the composition to rapidly dissipate static electric charges from its surface. The ionic liquid is generally a salt that has a low enough melting temperature so that it can be in the form of a liquid when melt processed with the liquid crystalline polymer. For example, the melting temperature of the ionic liquid may be about 400° C. or less, in some embodiments about 350° C. or less, in some embodiments from about 1° C. to about 100° C., and in some embodiments, from about 5° C. to about 50° C. The salt contains a cationic species and counterion. The cationic species contains a compound having at least one heteroatom (e.g., nitrogen or phosphorous) as a “cationic center.” Examples of such heteroatomic compounds include, for instance, quaternary oniums having the following structures:

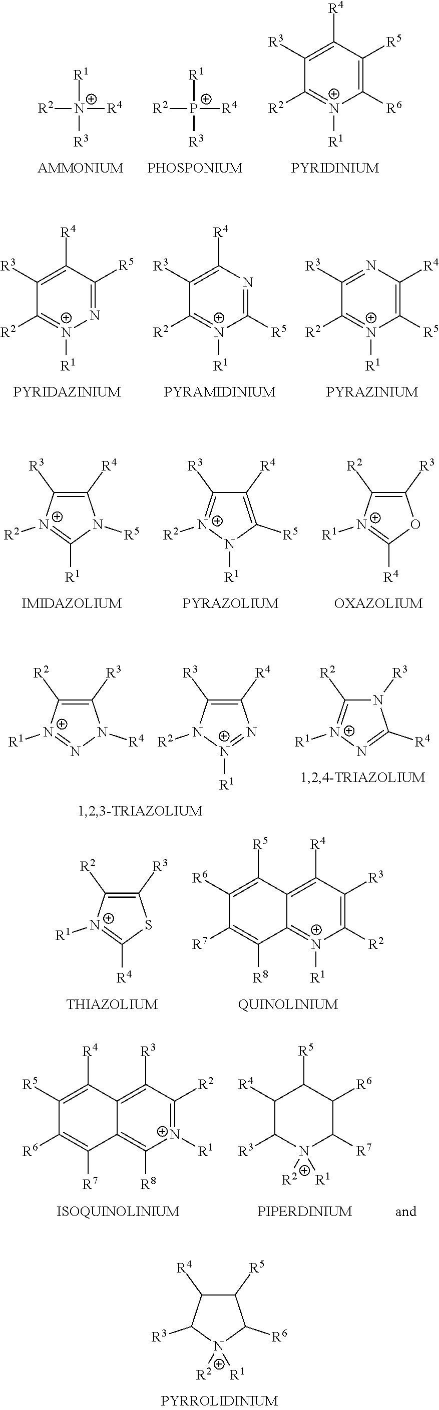

wherein, R¹, R², R³, R⁴, R⁵, R⁶, R⁷, and R⁸ are independently selected from the group consisting of hydrogen; substituted or unsubstituted C₁-C₁₀ alkyl groups (e.g., methyl, ethyl, n-propyl, isopropyl, n-butyl, isobutyl, sec-butyl, tert-butyl, n-pentyl, etc.); substituted or unsubstituted C₃-C₁₄ cycloalkyl groups (e.g., adamantyl, cyclopropyl, cyclobutyl, cyclopentyl, cyclooctyl, cyclohexenyl, etc.); substituted or unsubstituted C₁-C₁₀ alkenyl groups (e.g., ethylene, propylene, 2-methypropylene, pentylene, etc.); substituted or unsubstituted C₂-C₁₀ alkynyl groups (e.g., ethynyl, propynyl, etc.); substituted or unsubstituted C₁-C₁₀ alkoxy groups (e.g., methoxy, ethoxy, n-propoxy, isopropoxy, n-butoxy, t-butoxy, sec-butoxy, n-pentoxy, etc.); substituted or unsubstituted acyloxy groups (e.g., methacryloxy, methacryloxyethyl, etc.); substituted or unsubstituted aryl groups (e.g., phenyl); substituted or unsubstituted heteroaryl groups (e.g., pyridyl, furanyl, thienyl, thiazolyl, isothiazolyl, triazolyl, imidazolyl, isoxazolyl, pyrrolyl, pyrazolyl, pyridazinyl, pyrimidinyl, quinolyl, etc.); and so forth. In one particular embodiment, for example, the cationic species may be an ammonium compound having the structure N+R¹R²R³R⁴, wherein R¹, R², and/or R³ are independently a C₁-C₆ alkyl (e.g., methyl, ethyl, butyl, etc.) and R⁴ is hydrogen or a C₁-C₄ alkyl group (e.g., methyl or ethyl). For example, the cationic component may be tri-butylmethylammonium, wherein R¹, R², and R³ are butyl and R⁴ is methyl.

Suitable counterions for the cationic species may include, for example, halogens (e.g., chloride, bromide, iodide, etc.); sulfates or sulfonates (e.g., methyl sulfate, ethyl sulfate, butyl sulfate, hexyl sulfate, octyl sulfate, hydrogen sulfate, methane sulfonate, dodecylbenzene sulfonate, dodecylsulfate, trifluoromethane sulfonate, heptadecafluorooctanesulfonate sodium dodecylethoxysulfate, etc.); sulfosuccinates; amides (e.g., dicyanamide); imides (e.g., bis(pentafluoroethyl-sulfonyl)imide, bis(trifluoromethylsulfonyl)imide, bis(trifluoromethyl)imide, etc.); borates (e.g., tetrafluoroborate, tetracyanoborate, bis[oxalato]borate, bis[salicylato]borate, etc.); phosphates or phosphinates (e.g., hexafluorophosphate, diethylphosphate, bis(pentafluoroethyl)phosphinate, tris(pentafluoroethyl)-trifluorophosphate, tris(nonafluorobutyl)trifluorophosphate, etc.); antimonates (e.g., hexafluoroantimonate); alum inates (e.g., tetrachloroaluminate); fatty acid carboxylates (e.g., oleate, isostearate, pentadecafluorooctanoate, etc.); cyanates; acetates; and so forth, as well as combinations of any of the foregoing. To help improve compatibility with the liquid crystalline polymer, it may be desired to select a counterion that is generally hydrophobic in nature, such as imides, fatty acid carboxylates, etc. Particularly suitable hydrophobic counterions may include, for instance, bis(pentafluoroethylsulfonyl)imide, bis(trifluoromethylsulfonyl)imide, and bis(trifluoromethyl)imide.

When employed, electrically conductive fillers typically constitute from about 0.5 to about 20 parts, in some embodiments from about 1 to about 15 parts, and in some embodiments, from about 2 to about 8 parts by weight per 100 parts by weight of liquid crystalline polymers employed in the composition. For example, electrically conductive fillers may constitute from about 0.1 wt. % to about 10 wt. %, in some embodiments from about 0.2 wt. % to about 8 wt. %, and in some embodiments, from about 0.5 wt. % to about 4 wt. % of the polymer composition.

iii. Impact Modifier

An impact modifier may also be employed in the polymer composition. For example, the impact modifier may be a polymer that contains an olefinic monomeric unit that derived from one or more α-olefins. Examples of such monomers include, for instance, linear and/or branched α-olefins having from 2 to 20 carbon atoms and typically from 2 to 8 carbon atoms. Specific examples include ethylene, propylene, 1-butene; 3-methyl-1-butene; 3,3-dimethyl-1-butene; 1-pentene; 1-pentene with one or more methyl, ethyl or propyl substituents; 1-hexene with one or more methyl, ethyl or propyl substituents; 1-heptene with one or more methyl, ethyl or propyl substituents; 1-octene with one or more methyl, ethyl or propyl substituents; 1-nonene with one or more methyl, ethyl or propyl substituents; ethyl, methyl or dimethyl-substituted 1-decene; 1-dodecene; and styrene. Particularly desired α-olefin monomers are ethylene and propylene. The olefin polymer may be in the form of a copolymer that contains other monomeric units as known in the art. For example, another suitable monomer may include a “(meth)acrylic” monomer, which includes acrylic and methacrylic monomers, as well as salts or esters thereof, such as acrylate and methacrylate monomers. Examples of such (meth)acrylic monomers may include methyl acrylate, ethyl acrylate, n-propyl acrylate, i-propyl acrylate, n-butyl acrylate, s-butyl acrylate, i-butyl acrylate, t-butyl acrylate, n-amyl acrylate, i-amyl acrylate, isobornyl acrylate, n-hexyl acrylate, 2-ethylbutyl acrylate, 2-ethylhexyl acrylate, n-octyl acrylate, n-decyl acrylate, methylcyclohexyl acrylate, cyclopentyl acrylate, cyclohexyl acrylate, methyl methacrylate, ethyl methacrylate, 2-hydroxyethyl methacrylate, n-propyl methacrylate, n-butyl methacrylate, i-propyl methacrylate, i-butyl methacrylate, n-amyl methacrylate, n-hexyl methacrylate, i-amyl methacrylate, s-butyl-methacrylate, t-butyl methacrylate, 2-ethylbutyl methacrylate, methylcyclohexyl methacrylate, cinnamyl methacrylate, crotyl methacrylate, cyclohexyl methacrylate, cyclopentyl methacrylate, 2-ethoxyethyl methacrylate, isobornyl methacrylate, etc., as well as combinations thereof. In one embodiment, for instance, the impact modifier may be an ethylene methacrylic acid copolymer (“EMAC”). When employed, the relative portion of the monomeric component(s) may be selectively controlled. The α-olefin monomer(s) may, for instance, constitute from about 55 wt. % to about 95 wt. %, in some embodiments from about 60 wt. % to about 90 wt. %, and in some embodiments, from about 65 wt. % to about 85 wt. % of the copolymer. Other monomeric components (e.g., (meth)acrylic monomers) may constitute from about 5 wt. % to about 35 wt. %, in some embodiments from about 10 wt. % to about 32 wt. %, and in some embodiments, from about 15 wt. % to about 30 wt. % of the copolymer.

Other suitable suitable olefin copolymers may be those that are “epoxy-functionalized” in that they contain, on average, two or more epoxy functional groups per molecule. The copolymer may also contain an epoxy-functional monomeric unit. One example of such a unit is an epoxy-functional (meth)acrylic monomeric component. For example, suitable epoxy-functional (meth)acrylic monomers may include, but are not limited to, those containing 1,2-epoxy groups, such as glycidyl acrylate and glycidyl methacrylate. Other suitable epoxy-functional monomers include allyl glycidyl ether, glycidyl ethylacrylate, and glycidyl itoconate. Other suitable monomers may also be employed to help achieve the desired molecular weight. In one particular embodiment, for example, the copolymer may be a terpolymer formed from an epoxy-functional (meth)acrylic monomeric component, α-olefin monomeric component, and non-epoxy functional (meth)acrylic monomeric component. The copolymer may, for instance, be poly(ethylene-co-butylacrylate-co-glycidyl methacrylate). When employed, the epoxy-functional (meth)acrylic monomer(s) typically constitutes from about 1 wt. % to about 20 wt. %, in some embodiments from about 2 wt. % to about 15 wt. %, and in some embodiments, from about 3 wt. % to about 10 wt. % of the copolymer.

When employed, impact modifiers typically constitute from about 0.5 to about 60 parts, in some embodiments from about 1 to about 50 parts, and in some embodiments, from about 2 to about 30 parts by weight per 100 parts by weight of the liquid crystalline polymers employed in the composition. For example, impact modifiers may constitute from about 0.1 wt. % to about 30 wt. %, in some embodiments from about 0.5 wt. % to about 25 wt. %, and in some embodiments, from about 1 wt. % to about 20 wt. % of the polymer composition.

iv. Laser Activatable Additive

In some embodiments, the polymer composition may be “laser activatable” in the sense that it contains an additive that can be activated by a laser direct structuring (“LDS”) process to form conductive elements (e.g., antenna elements) thereon. In such a process, the additive is exposed to a laser that causes the release of metals. The laser thus draws the pattern of conductive elements onto the part and leaves behind a roughened surface containing embedded metal particles. These particles act as nuclei for the crystal growth during a subsequent plating process (e.g., copper plating, gold plating, nickel plating, silver plating, zinc plating, tin plating, etc.). When employed, laser activatable additives typically constitute from about 0.1 wt. % to about 30 wt. %, in some embodiments from about 0.5 wt. % to about 20 wt. %, and in some embodiments, from about 1 wt. % to about 10 wt. % of the polymer composition. The laser activatable additive generally includes spinel crystals, which may include two or more metal oxide cluster configurations within a definable crystal formation. For example, the overall crystal formation may have the following general formula:

AB₂O₄

wherein,

A is a metal cation having a valance of 2, such as cadmium, chromium, manganese, nickel, zinc, copper, cobalt, iron, magnesium, tin, titanium, etc., as well as combinations thereof; and

B is a metal cation having a valance of 3, such as chromium, iron, aluminum, nickel, manganese, tin, etc., as well as combinations thereof.

Typically, A in the formula above provides the primary cation component of a first metal oxide cluster and B provides the primary cation component of a second metal oxide cluster. These oxide clusters may have the same or different structures. In one embodiment, for example, the first metal oxide cluster has a tetrahedral structure and the second metal oxide cluster has an octahedral cluster. Regardless, the clusters may together provide a singular identifiable crystal type structure having heightened susceptibility to electromagnetic radiation. Examples of suitable spinel crystals include, for instance, MgAl₂O₄, ZnAl₂O₄, FeAl₂O₄, CuFe₂O₄, CuCr₂O₄, MnFe₂O₄, NiFe₂O₄, TiFe₂O₄, FeCr₂O₄, MgCr₂O₄, etc. Copper chromium oxide (CuCr₂O₄) is particularly suitable for use in the present invention and is available from Shepherd Color Co. under the designation “Shepherd Black 1GM.”

v. Other Additives

A wide variety of additional additives can also be included in the polymer composition, such as fibers (e.g., glass fibers), lubricants, thermally conductive fillers, pigments, antioxidants, stabilizers, surfactants, waxes, flame retardants, anti-drip additives, nucleating agents (e.g., boron nitride) and other materials added to enhance properties and processability. Lubricants, for example, may be employed in the polymer composition that are capable of withstanding the processing conditions of the liquid crystalline polymer without substantial decomposition. Examples of such lubricants include fatty acids esters, the salts thereof, esters, fatty acid amides, organic phosphate esters, and hydrocarbon waxes of the type commonly used as lubricants in the processing of engineering plastic materials, including mixtures thereof. Suitable fatty acids typically have a backbone carbon chain of from about 12 to about 60 carbon atoms, such as myristic acid, palmitic acid, stearic acid, arachic acid, montanic acid, octadecinic acid, parinric acid, and so forth. Suitable esters include fatty acid esters, fatty alcohol esters, wax esters, glycerol esters, glycol esters and complex esters. Fatty acid amides include fatty primary amides, fatty secondary amides, methylene and ethylene bisamides and alkanolamides such as, for example, palmitic acid amide, stearic acid amide, oleic acid amide, N,N′-ethylenebisstearamide and so forth. Also suitable are the metal salts of fatty acids such as calcium stearate, zinc stearate, magnesium stearate, and so forth; hydrocarbon waxes, including paraffin waxes, polyolefin and oxidized polyolefin waxes, and microcrystalline waxes. Particularly suitable lubricants are acids, salts, or amides of stearic acid, such as pentaerythritol tetrastearate, calcium stearate, or N,N′-ethylenebisstearamide. When employed, the lubricant(s) typically constitute from about 0.05 wt. % to about 1.5 wt. %, and in some embodiments, from about 0.1 wt. % to about 0.5 wt. % (by weight) of the polymer composition.

### II. Formation

The components used to form the polymer composition may be combined together using any of a variety of different techniques as is known in the art. In one particular embodiment, for example, the liquid crystalline polymer and other optional additives are melt processed as a mixture within an extruder to form the polymer composition. The mixture may be melt-kneaded in a single-screw or multi-screw extruder at a temperature of from about 200° C. to about 450° C. In one embodiment, the mixture may be melt processed in an extruder that includes multiple temperature zones. The temperature of individual zones is typically set within about −60° C. to about 25° C. relative to the melting temperature of the polymer. By way of example, the mixture may be melt processed using a twin screw extruder such as a Leistritz 18-mm co-rotating fully intermeshing twin screw extruder. A general purpose screw design can be used to melt process the mixture. In one embodiment, the mixture including all of the components may be fed to the feed throat in the first barrel by means of a volumetric feeder. In another embodiment, different components may be added at different addition points in the extruder, as is known. For example, the polymer may be applied at the feed throat, and certain additives (e.g., particulate filler) may be supplied at the same or different temperature zone located downstream therefrom. Regardless, the resulting mixture can be melted and mixed then extruded through a die. The extruded polymer composition can then be quenched in a water bath to solidify and granulated in a pelletizer followed by drying.

### III. Shaped Part

As will be described in more detail below, the polymer composition may be used to form various parts of the PPG sensor, such as an insulative portion of the light source (e.g., LED) and/or the optical detector (e.g., photodetector), the housing of the sensor, substrates for antenna components, etc. Due in part to the beneficial properties of the polymer composition, the part may have a very small size, such as a thickness of about 5 millimeters or less, in some embodiments about 4 millimeters or less, in some embodiments from about 0.1 to about 3.5 millimeters, and in some embodiments, from about 0.5 to about 3 millimeters. Regardless of the specific part in which it is employed, the polymer composition is generally shaped into the desired part using a variety of different techniques. Suitable techniques may include, for instance, film extrusion, thermoforming, injection molding, low-pressure injection molding, extrusion compression molding, gas injection molding, foam injection molding, low-pressure gas injection molding, low-pressure foam injection molding, gas extrusion compression molding, foam extrusion compression molding, extrusion molding, foam extrusion molding, compression molding, foam compression molding, gas compression molding, etc. For example, an injection molding system may be employed that includes a mold within which the polymer composition may be injected. The time inside the injector may be controlled and optimized so that polymer matrix is not pre-solidified. When the cycle time is reached and the barrel is full for discharge, a piston may be used to inject the composition to the mold cavity. Compression molding systems may also be employed. As with injection molding, the shaping of the polymer composition into the desired article also occurs within a mold. The composition may be placed into the compression mold using any known technique, such as by being picked up by an automated robot arm. The temperature of the mold may be maintained at or above the solidification temperature of the polymer composition for a desired time period to allow for solidification. The molded product may then be solidified by bringing it to a temperature below that of the melting temperature. The resulting product may be de-molded. The cycle time for each molding process may be adjusted to suit the polymer composition, to achieve sufficient bonding, and to enhance overall process productivity.

Depending on the manner in which it is used in the PPG sensor (e.g., antenna element), it may also be desired to deposit one or more conductive elements on the molded part using any of a variety of known metal deposition techniques, such as plating (e.g., electrolytic plating, electroless plating, etc.), printing (e.g., digital printing, aerosol jet printing, etc.), and so forth. The conductive elements may contain one or more of a variety of conductive materials, such as a metal, e.g., gold, silver, nickel, aluminum, copper, as well as mixture or alloys thereof. In one embodiment, for instance, the conductive elements may include copper and/or nickel (e.g., pure or alloys thereof). If desired, a seed layer may initially be formed on the substrate to facilitate the metal deposition process.

When plating is employed as a deposition technique, the process may vary as desired. In certain embodiments, for instance, the process may include initially forming a pattern on the surface of the molded part based on the desired circuit interconnect pattern. This may be accomplished using various known techniques, such as laser ablation or patterning, plasma etching, ultraviolet light treatment, acid etching, etc. Regardless, after forming the desired pattern on the molded part, the patterned regions may then optionally be subjected to an activation process to prepare for subsequent metal deposition. During this process, the patterned substrate may be contacted with an activation solution that contains a metal, such as palladium, platinum, iridium, rhodium, etc., as well as mixtures thereof. Palladium is particularly suitable. Once the surface has been conditioned as described above, a first metal layer may then be formed thereon on the patterned substrate, such as through a process known as electroless plating. Electroless plating may occur through auto-catalytic reactions in which the metal deposited on the surface acts as a catalyst for further depositing. Typically, nickel and/or copper are electrolessly plated onto the surface of the patterned substrate. Electroless nickel plating may be accomplished, for example, using a solution that contains a nickel salt (e.g., nickel sulfate). If desired, the patterned substrate may also be subjected to one or more additional steps to form the final metal coating layer(s). Additional coating layer(s) are typically deposited using a process known as electrolytic plating, during which the patterned substrate is contacted with a metal solution and subjected to an electrical current to initiate deposition of the metal. For example, a second metal layer may be electrolytically deposited over the first metal layer (e.g., electrolessly plated copper and/or nickel). The second metal layer may include, for instance, copper or nickel. In certain embodiments, one or more additional metal layer(s), such as copper and/or nickel, may also be electrolytically deposited over the second metal layer.

### IV. PPG Sensor

As indicated above, the polymer composition of the present invention is employed in a PPG sensor. While the particular configuration of the sensor may vary as is known to those skilled in the art, it generally includes at least one light source and at least one optical detector. A PPG signal (derived from the amount of light reflected after interaction with tissue) obtained from received light can be processed to obtain physiological information (e.g., heart rate, oxygen saturation, etc.). Suitable light sources may include, for instance, light emitting diodes (LEDs), incandescent lights, fluorescent lights, etc. When employed, the LED may be a green LED, red LED, an infrared (IR) LED, charged couple devices, etc. When more than one light source is employed, the same or different light sources (with different emission wavelengths) may be employed. For example, a combination of one or more green LEDs and IR LEDs may be used. Suitable optical detectors (or photodetectors) may include, for instance, photodiodes, phototransistors, etc. The light source(s) may emit light having one or more wavelengths that are specific or directed to a type of physiological data to be collected. In one embodiment, the light source(s) may emit light having one or more wavelengths specific to the collection of PPG signals. Similarly, the optical detector(s) may sample, measure and/or detect one or more wavelengths that are also specific or directed to a type of physiological data to be collected. For instance, a light source emitting light having a wavelength in the green spectrum and an optical detector positioned to detect a response or reflection corresponding with such light may provide data that may be used to determine, such as heart rate and oxygen saturation. In certain embodiments of the present invention, the polymer composition may be used to form a portion (e.g., housing or insulative portion) of the light source(s), optical detector(s), or a combination thereof.

Because the optical detector is typically sensitive to light arriving from all arrival angles, e.g., light including non-pulsatile signal artifacts and light including pulsatile signals, one or more viewing components may also be employed in the PPG sensor to improve the PPG signal by substantially receiving light reflected from a tissue layer containing blood vessels. More particularly, such viewing component(s) may be configured to receive light reflected at certain viewing angles associated with reflection from a pulsatile signal tissue layer, and thus, a high or maximum perfusion index. When employed, the viewing component(s) are generally coupled to the photodetector of the PPG sensor. When a plurality of viewing components is employed, they may be the same or different viewing components. Additionally, each one of the plurality of viewing components can have a unique viewing angle, or the plurality of viewing components can have the same viewing angle. In one variation, the viewing component can be attached to the photodetector using conventional methods, e.g., by the use of adhesives, welding, etc. Some variations of the viewing component may likewise include light tubes (or light pipes) angled to allow light to be received at viewing angles associated with a high level of pulsatile signal, and block light travelling at other angles to the photodetector. One or more light tubes may be coupled to the PPG sensor, typically to the photodetector. In certain embodiments, the polymer composition may be used to form a portion (e.g., insulative portion) of the viewing component(s). A housing may also enclose the light source(s), optical detector(s), and/or viewing component(s), which may optionally be formed from the polymer composition.

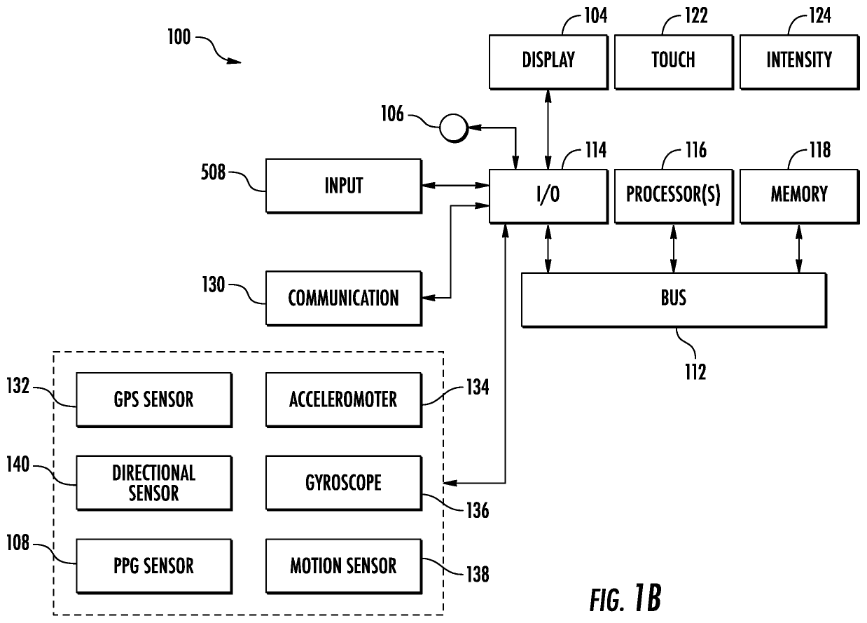

In some examples, the PPG sensor can be configured with a combination of the same and different wavelengths of light, the same and different separation distances, and the same and different viewing angles. FIG. 2 illustrates an exemplary PPG sensor according to examples of the disclosure. The PPG sensor can include a plurality of LEDs, such as LED 700 and LED 701, and a plurality of photodetectors, such as photodetectors 702, 704, 706, and 708. LED 700 and LED 701 can be configured to measure emit wavelengths of light. For example, LED 700 can be configured to emit infrared wavelengths, while LED 701 can be configured to emit green wavelengths. Photodetector 704 can be configured to measure light emitted by LED 700, and photodetector 708 can be configured to measure light emitted by LED 701. Photodetectors 702 and 706 can be configured to measure light emitted by both LED 700 and LED 701. Photodetector 702 can be located a separation distance d10 from LED 700 and LED 701; photodetector 704 can be located a separation distance d7 from LED 700; photodetector 706 can be located a separation distance d8 from LED 700 and LED 701; and photodetector 708 can be located a separation distance d9 from LED 701. In some examples, separation distance d8 can be the same as separation distance d10. In some examples, separation distance d7 can be different from separation distance d9. Additionally or alternatively, separation distance d8 (or separation distance d10) can be different from separation distance d7 (and/or separation distance d9). A viewing component 712, 714, 716, and 718 be coupled to each photodetector 702, 704, 706, and 708, respectively. The viewing components can be configured with the same or different viewing angle(s).

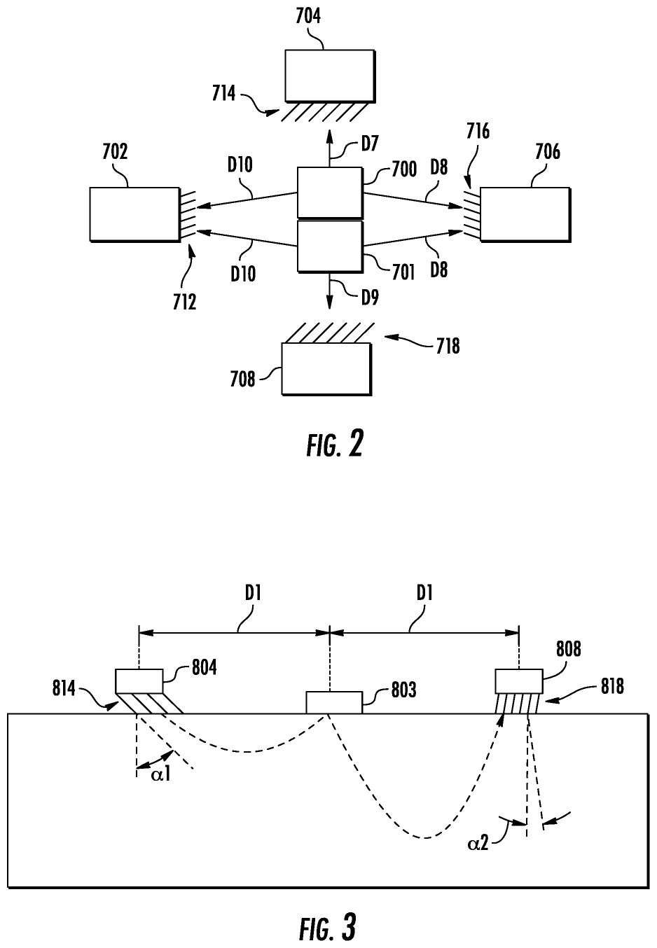

FIG. 3 illustrates another embodiment of the PPG sensor according to the present invention. In this embodiment, the PPG sensor can include LED 803 and photodetectors 804 and 808. Photodetectors 804 and 808 can have the same separation distance “Dl” from LED 803. Photodetector 804 can be coupled to viewing component 814, and photodetector 808 can be coupled to viewing component 818. In some examples, a viewing component 814 can be configured with different viewing angle(s) “α₁” than the viewing angle(s) “α₂” of viewing component 818. Photodetector 808 and viewing component 818 can measure a different depth than photodetector 804 and viewing component 814. The controller can evaluate the strength of physiological signal (e.g., using frequency spectrum analysis like FFT) to select one or both signals for processing. Based on the selection, in some examples, the controller can continue to use the selected photodetector(s) for subsequent measurements.

In some embodiments, information/data obtained by the PPG sensor may be transmitted to a remote location, e.g., a computer, doctor's office, etc. In such embodiments, the PPG sensor may contain an antenna system that includes a circuit structure. The circuit structure may, for instance, contain a substrate on which is disposed on or more antenna elements. If desired, the substrate may be formed from the polymer composition of the present invention. The circuit structure may be configured to transmit radiofrequency signals at a variety of frequencies, including Wireless Medical Telemetry Service (WMTS), Bluetooth, LTE, and 5G frequencies. For example, the circuit structure may be particularly well suited for 5G systems. As used herein, “5G” generally refers to high speed data communication over radiofrequency signals. 5G networks and systems are capable of communicating data at much faster rates than previous generations of data communication standards (e.g., “4G, “LTE”). For example, as used herein, “5G frequencies” can refer to frequencies that are 1.5 GHz or more, in some embodiments about 2.0 GHz or more, in some embodiments about 2.5 GHz or higher, in some embodiments about 3.0 GHz or higher, in some embodiments from about 3 GHz to about 300 GHz, or higher, in some embodiments from about 4 GHz to about 80 GHz, in some embodiments from about 5 GHz to about 80 GHz, in some embodiments from about 20 GHz to about 80 GHz, and in some embodiments from about 28 GHz to about 60 GHz. Various standards and specifications have been released quantifying the requirements of 5G communications. As one example, the International Telecommunications Union (ITU) released the International Mobile Telecommunications-2020 (“IMT-2020”) standard in 2015. The IMT-2020 standard specifies various data transmission criteria (e.g., downlink and uplink data rate, latency, etc.) for 5G. The IMT-2020 Standard defines uplink and downlink peak data rates as the minimum data rates for uploading and downloading data that a 5G system must support. The IMT-2020 standard sets the downlink peak data rate requirement as 20 Gbit/s and the uplink peak data rate as 10 Gbit/s. As another example, 3rd Generation Partnership Project (3GPP) recently released new standards for 5G, referred to as “5G NR.” 3GPP published “Release 15” in 2018 defining “Phase 1” for standardization of 5G NR. 3GPP defines 5G frequency bands generally as “Frequency Range 1” (FR1) including sub-6 GHz frequencies and “Frequency Range 2” (FR2) as frequency bands ranging from 20-60 GHz. Antenna systems described herein can satisfy or qualify as “5G” under standards released by 3GPP, such as Release 15 (2018), and/or the IMT-2020 Standard.

To achieve high speed data communication at high frequencies, antenna elements and arrays may employ small feature sizes/spacing (e.g., fine pitch technology) that can improve antenna performance. For example, the feature size (spacing between antenna elements, width of antenna elements) etc. is generally dependent on the wavelength (“λ”) of the desired transmission and/or reception radiofrequency propagating through the substrate dielectric on which the antenna element is formed (e.g., nλ/4 where n is an integer and λ is dependent on the dielectric constant of the substrate). Further, beamforming and/or beam steering can be employed to facilitate receiving and transmitting across multiple frequency ranges or channels (e.g., multiple-in-multiple-out (MIMO), massive MIMO). The high frequency 5G antenna elements can have a variety of configurations. For example, the 5G antenna elements can be or include co-planar waveguide elements, patch arrays (e.g., mesh-grid patch arrays), other suitable 5G antenna configurations. The antenna elements can be configured to provide MIMO, massive MIMO functionality, beam steering, and the like. As used herein “massive” MIMO functionality generally refers to providing a large number transmission and receiving channels with an antenna array achieved by frequency domain and/or time domain multiplexing, for example 8 transmission (Tx) and 8 receive (Rx) channels (abbreviated as 8×8). Massive MIMO functionality may be provided with 8×8, 12×12, 16×16, 32×32, 64×64, or greater.

The antenna elements can have a variety of configurations and arrangements and can be fabricated using a variety of manufacturing techniques. As one example, the antenna elements and/or associated elements (e.g., ground elements, feed lines, etc.) can employ fine pitch technology. Fine pitch technology generally refers to small or fine spacing between their components or leads. For example, feature dimensions and/or spacing between antenna elements (or between an antenna element and a ground plane) can be about 1,500 micrometers or less, in some embodiments 1,250 micrometers or less, in some embodiments 750 micrometers or less (e.g., center-to-center spacing of 1.5 mm or less), 650 micrometers or less, in some embodiments 550 micrometers or less, in some embodiments 450 micrometers or less, in some embodiments 350 micrometers or less, in some embodiments 250 micrometers or less, in some embodiments 150 micrometers or less, in some embodiments 100 micrometers or less, and in some embodiments 50 micrometers or less. However, it should be understood that feature sizes and/or spacings that are smaller and/or larger may be employed within the scope of this disclosure. As a result of such small feature dimensions, antenna systems can be achieved with a large number of antenna elements in a small footprint. For example, an antenna array can have an average antenna element concentration of greater than 1,000 antenna elements per square centimeter, in some embodiments greater than 2,000 antenna elements per square centimeter, in some embodiments greater than 3,000 antenna elements per square centimeter, in some embodiments greater than 4,000 antenna elements per square centimeter, in some embodiments greater than 6,000 antenna elements per square centimeter, and in some embodiments greater than about 8,000 antenna elements per square centimeter. Such compact arrangement of antenna elements can provide a greater number of channels for MIMO functionality per unit area of the antenna area. For example, the number of channels can correspond with (e.g., be equal to or proportional with) the number of antenna elements.

The PPG sensor may be employed in a wide variety of electronic components. In one suitable configuration, the PPG sensor may be employed in a portable electronic component in which the available interior space is relatively small. Examples of suitable portable electronic components include cellular telephones, laptop computers, small portable computers (e.g., ultraportable computers, netbook computers, and tablet computers), wrist-watch devices, pendant devices, headphone and earpiece devices, media players with wireless communications capabilities, handheld computers (also sometimes called personal digital assistants), remote controllers, global positioning system (GPS) devices, handheld gaming devices, etc.

In one particular embodiment, the PPG sensor is employed in a wearable device. The wearable device may be any electronic device suitable for contact with a body region of an individual, e.g., a user's skin, wrist, arm, leg, finger, etc. Accordingly, the wearable device may be a phone, wristwatch, arm or wristband, headband, or any wearable device suitable for collecting PPG signals or biometric information. The wearable device may be worn on a wrist, ankle, head, chest, leg, finger, etc., with the use of a band that is flexible and capable of adjustably fitting a user, or it may be rigidly sized to fit. In one embodiment, the wearable device is a wristwatch. As noted above, the wearable device may be generally used to measure PPG signals from a user. The PPG signals may then be used to extrapolate and monitor various types of physiological information/data. In some variations, the PPG signal is used to obtain information related to the heart rate of a user. The acquisition of a PPG signal related to, e.g., heart rate or heart rate variation, may be indicated to the user on the display of the wearable device. Heart rate may be indicated in any suitable manner by the wearable device. For example, heart rate may be indicated as a numerical value, a picture, or text on the device display, or be audibly provided by the wearable device. Heart rate may be indicated by combinations of any of the foregoing. In some variations, the wearable device may include a signal-strength indicator that is represented by the pulsing of an LED viewable by the user. Some implementations of the wearable device may use a light such as an LED to display the heart rate of the user by modulating the amplitude of the light emitted at the frequency of the user's heart rate. Other types of physiological data may be indicated in the same manner. Notifications relating to the obtained heart rate or other physiological data can also indicated in the same manner.

Generally speaking, the wearable device may include a housing structured for attachment to a body region of the individual. If desired, all or a part of the housing may also be formed from the polymer composition of the present invention. Regardless, the housing receives the PPG sensor and optionally a processor configured to analyze the PPG signal obtained from the PPG sensor and determine the physiological parameter. The housing may be configured to have any size and shape suitable for the body region of contact, and may include a housing comprising an upper surface, a back surface, and side surfaces, an interior enclosed within the surfaces, and a display that is mounted in the upper surface of the housing. The display may, for example, be a touch screen that incorporates capacitive touch electrodes or may be a display that is not touch sensitive. The display may include image pixels formed from light-emitting diodes (LEDs), organic LEDs (OLEDs), plasma cells, electrowetting pixels, electrophoretic pixels, liquid crystal display (LCD) components, or other suitable image pixel structures. A display cover layer (e.g., glass or transparent plastic) may optionally cover the surface of display. An antenna system, such as described above, may also be employed within the wearable device. The wearable device may also include a power system for powering the various components. The power system may include a power management system, one or more power sources (e.g., battery, alternating current (AC)), a recharging system, a power failure detection circuit, a power converter or inverter, a power status indicator (e.g., a light-emitting diode (LED)) and any other components associated with the generation, management, and distribution of power in portable devices.

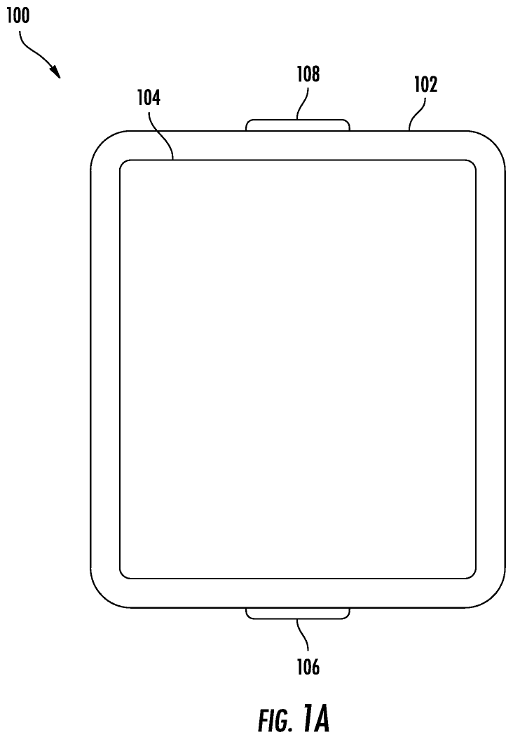

Referring to FIG. 1A, for example, one embodiment of a suitable wearable device 100 is shown in more detail. In this embodiment, the wearable device 100 includes a housing 102 and a touch-sensitive display screen 104. The touch screen 104 (or the touch-sensitive surface) may have one or more intensity sensors for detecting intensity of contacts (e.g., touches) being applied. The one or more intensity sensors of touch screen 104 can provide output data that represents the intensity of touches. The user interface of device 100 can respond to touches based on their intensity, meaning that touches of different intensities can invoke different user interface operations on device 100. In some embodiments, the device 100 may also have one or more input mechanisms 106 and 508, such as push buttons or rotatable mechanisms. The device 100 may also include one or more attachment mechanisms, such as bands that may be secured to the user through the use of hooks and loops (e.g., Velcro), a clasp, and/or a band having memory of its shape, e.g., through the use of a spring metal band.

FIG. 1B provides further examples of the components that may be employed in the wearable device 100. More particularly, in this embodiment, the device 100 has a bus 112 that operatively couples I/O section 114 with one or more computer processors 116 and memory 118. I/O section 114 can be connected to the display 104, which can have touch-sensitive component 122 and, optionally, touch-intensity sensitive component 124. In addition, I/O section 114 can be connected with a communication unit 130 for receiving application and operating system data, such as an antenna system as described above that transmits signal by using Wi-Fi, Bluetooth, near field communication (NFC), cellular, and/or other wireless communication techniques. The wearable device 100 can also include various sensors, such as GPS sensor 132, accelerometer 134, directional sensor 140 (e.g., compass), gyroscope 136, motion sensor 138, PPG sensor 108, all of which can be operatively connected to I/O section 114. The memory 118 can be a non-transitory computer-readable storage medium, for storing computer-executable instructions, which, when executed by one or more computer processors 116, for example, can cause the computer processors to perform the algorithms further described below. The computer-executable instructions can also be stored and/or transported within any non-transitory computer-readable storage medium for use by or in connection with an instruction execution system, apparatus, or device, such as a computer-based system, processor-containing system, or other system that can fetch the instructions from the instruction execution system, apparatus, or device and execute the instructions. For purposes of this document, a “non-transitory computer-readable storage medium” can be any medium that can tangibly contain or store computer-executable instructions for use by or in connection with the instruction execution system, apparatus, or device. The non-transitory computer-readable storage medium can include, but is not limited to, magnetic, optical, and/or semiconductor storages. A processor (not shown) may be included in the housing that is configured to run various algorithms based on information obtained from the PPG sensor.

The PPG sensor may be provided in any suitable location of the wearable device. In one embodiment, the housing of the wearable device includes the PPG sensor as noted above. The PPG sensor may be provided a distinct device that is contained within the housing, such as on the back housing surface. In other embodiments, however, the components of the PPG sensor (e.g., light source(s) and/or optical detector(s)) may be provided within the housing as separate components that communicate together to form an integrated PPG sensor system. For example, the light source(s) may be provided on a combination of surfaces, such as the back surface and a side surface of the wearable device housing. One or more light sources may also be provided on an attachment mechanism of the wearable device. For example, when the wearable device is a wristwatch, one or more of the light sources may be disposed on the wristband. The optical detectors may likewise be provided and arranged on any suitable location(s) of the wearable device. In one embodiment, one or more detectors may be disposed on the back surface of the wearable device housing. In another embodiment, one or more optical detectors may be disposed on a side surface of the wearable device housing. One or more detectors may likewise be provided on an attachment mechanism of the wearable device. For example, when the wearable device is a wristwatch, one or more of the detectors can be disposed on the wristband.

The following test methods may be employed to determine certain of the properties referenced herein.

Test Methods

Melt Viscosity: The melt viscosity (Pa-s) may be determined in accordance with ISO Test No. 11443:2014 at a shear rate of 1,000 s⁻¹ and temperature 15° C. above the melting temperature using a Dynisco LCR7001 capillary rheometer. The rheometer orifice (die) had a diameter of 1 mm, length of 20 mm, L/D ratio of 20.1, and an entrance angle of 180°. The diameter of the barrel was 9.55 mm+0.005 mm and the length of the rod was 233.4 mm.

Melting Temperature: The melting temperature (“Tm”) may be determined by differential scanning calorimetry (“DSC”) as is known in the art. The melting temperature is the differential scanning calorimetry (DSC) peak melt temperature as determined by ISO Test No. 11357-2:2020. Under the DSC procedure, samples were heated and cooled at 20° C. per minute as stated in ISO Standard 10350 using DSC measurements conducted on a TA Q2000 Instrument.

Deflection Temperature Under Load (“DTUL”): The deflection under load temperature may be determined in accordance with ISO Test No. 75-2:2013 (technically equivalent to ASTM D648-18). More particularly, a test strip sample having a length of 80 mm, thickness of 10 mm, and width of 4 mm may be subjected to an edgewise three-point bending test in which the specified load (maximum outer fibers stress) was 1.8 Megapascals. The specimen may be lowered into a silicone oil bath where the temperature is raised at 2° C. per minute until it deflects 0.25 mm (0.32 mm for ISO Test No. 75-2:2013).

Tensile Modulus, Tensile Stress, and Tensile Elongation: Tensile properties may be tested according to ISO Test No. 527:2019 (technically equivalent to ASTM D638-14). Modulus and strength measurements may be made on the same test strip sample having a length of 80 mm, thickness of 10 mm, and width of 4 mm. The testing temperature may be 23° C., and the testing speeds may be 1 or 5 mm/min.

Flexural Modulus, Flexural Stress, and Flexural Elongation: Flexural properties may be tested according to ISO Test No. 178:2019 (technically equivalent to ASTM D790-10). This test may be performed on a 64 mm support span. Tests may be run on the center portions of uncut ISO 3167 multi-purpose bars. The testing temperature may be 23° C. and the testing speed may be 2 mm/min.

Charpy Impact Strength: Charpy properties may be tested according to ISO Test No. ISO 179-1:2010) (technically equivalent to ASTM D256-10, Method B). This test may be run using a Type 1 specimen size (length of 80 mm, width of 10 mm, and thickness of 4 mm). When testing the notched impact strength, the notch may be a Type A notch (0.25 mm base radius). Specimens may be cut from the center of a multi-purpose bar using a single tooth milling machine. The testing temperature may be 23° C.

Rockwell Hardness: Rockwell hardness is a measure of the indentation resistance of a material and may be determined in accordance with ASTM D785-08 (Scale M). Testing is performed by first forcing a steel ball indentor into the surface of a material using a specified minor load. The load is then increased to a specified major load and decreased back to the original minor load. The Rockwell hardness is a measure of the net increase in depth of the indentor, and is calculated by subtracting the penetration divided by the scale division from 130.

Dielectric Constant (“Dk”) and Dissipation Factor (“Df”): The dielectric constant (or relative static permittivity) and dissipation factor (or loss tangent) are determined at a frequency of 2 GHz in accordance with IPC 650 Test Method No. 2.5.5.13 (1/07). According to this method, the in-plane dielectric constant and dissipation factor may be determined using a split-cylinder resonator. The tested sample had a thickness of 8.175 mm, width of 70 mm, and length of 70 mm.

Surface/Volume Resistivity: The surface and volume resistivity values may be determined in accordance with IEC 62631-3-1:2016 or ASTM D257-14. According to this procedure, a standard specimen (e.g., 1 meter cube) is placed between two electrodes. A voltage is applied for sixty (60) seconds and the resistance is measured. The surface resistivity is the quotient of the potential gradient (in V/m) and the current per unit of electrode length (in Nm), and generally represents the resistance to leakage current along the surface of an insulating material. Because the four (4) ends of the electrodes define a square, the lengths in the quotient cancel and surface resistivities are reported in ohms, although it is also common to see the more descriptive unit of ohms per square. Volume resistivity is also determined as the ratio of the potential gradient parallel to the current in a material to the current density. In SI units, volume resistivity is numerically equal to the direct-current resistance between opposite faces of a one-meter cube of the material (ohm-m or ohm-cm).

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A photoplethysmographic sensor comprising a light source for emitting light onto tissue and an optical detector for receiving light that interacts with the tissue, wherein the sensor comprises a liquid crystalline polymer.

2. The photoplethysmographic sensor of claim 1, wherein the polymer composition exhibits a melt viscosity of 200 Pa-s or less as determined at a shear rate of 400 seconds⁻¹ and at a temperature 15° C. higher than the melting temperature of the composition in accordance with ISO Test No. 11443:2014.

3. The photoplethysmographic sensor of claim 1, wherein the polymer composition exhibits a tensile modulus of about 8,000 MPa or more as determined in accordance with ISO Test No. 527:2019.

4. The photoplethysmographic sensor of claim 1, wherein the polymer composition exhibits a tensile strength of from about 150 MPa or more as determined in accordance with ISO Test No. 527:2019.

5. The photoplethysmographic sensor of claim 1, wherein the polymer composition exhibits a flexural modulus of about 10,000 MPa or more as determined in accordance with ISO Test No. 178:2019 at 23° C.

6. The photoplethysmographic sensor of claim 1, wherein the polymer composition exhibits a Rockwell surface hardness of about 65 or less as determined in accordance with ASTM D785-08 (Scale M).

7. The photoplethysmographic sensor of claim 1, wherein the polymer composition exhibits a Charpy unnotched impact strength of about 45 kJ/m² or more as determined at 23° C. according to ISO Test No. 179-1:2010.

8. The photoplethysmographic sensor of claim 1, wherein the liquid crystalline polymer has a melting temperature of about 280° C. or more.

9. The photoplethysmographic sensor of claim 1, wherein the polymer composition exhibits a dissipation factor of about 0.01 or less at a frequency of 2 GHz.

10. The photoplethysmographic sensor of claim 1, wherein the polymer composition exhibits a dielectric constant of about 6 or less at a frequency of 2 GHz.

11. The photoplethysmographic sensor of claim 1, wherein the polymer composition exhibits a dielectric constant of greater than about 6 at a frequency of 2 GHz.

12. The photoplethysmographic sensor of claim 1, wherein the polymer composition exhibits an electromagnetic interference shielding effectiveness of about 20 decibels or more at a frequency of 2 GHz.

13. The photoplethysmographic sensor of claim 1, wherein the liquid crystalline polymer contains one or more repeating units derived from a hydroxycarboxylic acid.

14. The photoplethysmographic sensor of claim 13, wherein the hydroxycarboxylic acid repeating units constitute about 50 mol. % or more of the polymer.

15. The photoplethysmographic sensor of claim 13, wherein the liquid crystalline polymer contains repeating units derived from 4-hydroxybenzoic acid, 6-hydroxy-2-naphtoic acid, or a combination thereof.

16. The photoplethysmographic sensor of claim 15, wherein the liquid crystalline polymer further contains repeating units derived from terephthalic acid, isophthalic acid, 2,6-naphthalenedicarboxylic acid, hydroquinone, 4,4′-biphenol, acetaminophen, 4-aminophenol, or a combination thereof.

17. The photoplethysmographic sensor of claim 1, wherein the polymer composition comprises a mineral filler.

18. The photoplethysmographic sensor of claim 17, wherein the mineral filler includes particles.

19. The photoplethysmographic sensor of claim 18, wherein the particles include talc, mica, barium sulfate, or a combination thereof.

20. The photoplethysmographic sensor of claim 17, wherein the mineral filler includes fibers.

21. The photoplethysmographic sensor of claim 20, wherein the fibers include wollastonite.

22. The photoplethysmographic sensor of claim 1, wherein the polymer composition includes glass fibers.

23. The photoplethysmographic sensor of claim 1, wherein the polymer composition includes an impact modifier.

24. The photoplethysmographic sensor of claim 1, wherein the polymer composition includes an electrically conductive filler.

25. The photoplethysmographic sensor of claim 1, wherein the polymer composition includes a laser activatable additive.

26. The photoplethysmographic sensor of claim 1, wherein the light source contains the polymer composition.

27. The photoplethysmographic sensor of claim 1, wherein the optical detector contains the polymer composition.

28. The photoplethysmographic sensor of claim 1, further comprising a housing that encloses the light source and the optical detector, wherein the housing comprises the polymer composition.

29. The photoplethysmographic sensor of claim 1, wherein the light source includes a light emitting diode and the optical detector includes a photodiode.

30. The photoplethysmographic sensor of claim 1, wherein the sensor comprises a plurality of light sources and a plurality of optical detectors.

31. The photoplethysmographic sensor of claim 1, wherein the sensor comprises a viewing component that is coupled to the optical detector and is configured to receive light reflected from the tissue.

32. A wearable device comprising the photoplethysmographic sensor of claim 1.

33. The wearable device of claim 32, wherein the device is a wristwatch.

34. The wearable device of claim 32, wherein the wearable device comprises an attachment mechanism and a housing that encloses the optical detector and the light source.

35. The wearable device of claim 34, wherein the optical detector is disposed on a surface of the attachment mechanism, housing, or a combination thereof.

36. The wearable device of claim 34, wherein the light source is disposed on a surface of the attachment mechanism, housing, or a combination thereof.
